(19) United States
(12) Patent Application Publication
(10) Pub. No.: US 2015/0238654 A1
(43) Pub. Date: Aug. 27, 2015

(54) IRRIGATION RESISTANT COMPOSITIONS FOR REGENERATION OF HARD TISSUES AND METHODS AND KITS OF USING THE SAME

(71) Applicants: Gregory J. Pomrink, Newberry, FL (US); Zehra Tosun, Gainesville, FL (US)

(72) Inventors: Gregory J. Pomrink, Newberry, FL (US); Zehra Tosun, Gainesville, FL (US)

(21) Appl. No.: 14/695,910

(22) Filed: Apr. 24, 2015

Related U.S. Application Data
(63) Continuation-in-part of application No. 14/369,119, filed on Jun. 26, 2014.
(60) Provisional application No. 61/738,585, filed on Dec. 18, 2012.
(60) Provisional application No. 61/787,827, filed on Mar. 15, 2013.

(51) Int. Cl. A61L 27/10; A61L 27/46; A61B 17/88; A61L 27/54

(52) U.S. Cl. CPC A61L 27/10; A61L 27/54; A61L 27/46; A61B 17/8805; A61L 2430/02; A61L 2400/06; A61L 2300/112; A61L 2300/412

(57) ABSTRACT

An irrigation resistant bone repair composition including a biocompatible or bioactive bone repair material and at least one non-ionic surfactant, other than a non-random poly(oxyalkylene) block copolymer, is described. Also, methods for treating a bone having a bone gap or a bone defect with the composition including a biocompatible or bioactive bone repair material and at least one non-ionic surfactant, other than a non-random poly(oxyalkylene) block copolymer, are also provided. Also, kits including the irrigation resistant bone repair composition including a biocompatible or bioactive bone repair material and at least one non-ionic surfactant, other than a non-random poly(oxyalkylene) block copolymer, are described.

### RELATED APPLICATIONS

The present patent document is a continuation-in-part application of U.S. patent application Ser. No. 14/369,119, filed Jun. 26, 2014, which is §371 nationalization of International Application No. PCT/US2013/075741, filed Dec. 17, 2013, which claims the benefit of the filing date under 35 U.S.C. §119(e) of Provisional U.S. Patent Application Ser. Nos. 61/738,585, filed Dec. 18, 2012 and 61/787,827, filed Mar. 15, 2013, which are incorporated herein by reference in their entirety.

### BACKGROUND

Bone is a composite of collagen, cells, calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. There are many materials used today for the repair and regeneration of bone defects. For example, one material useful to encourage such repair and regeneration is bioactive glass.

Bioactive glass was originally developed in 1969 by L. Hench. Additionally, bioactive glasses were developed as bone replacement materials, with studies showing that bioactive glass can induce or aid in osteogenesis (Hench et al., J. Biomed. Mater. Res. 5:117-141 (1971)). Bioactive glass can form strong and stable bonds with bone (Piotrowski et al., J. Biomed. Mater. Res. 9:47-61(1975)). Further, bioactive glass is not considered toxic to bone or soft tissue from studies of in vitro and in vivo models (Wilson et al., J. Biomed. Mater. Res. 805-817 (1981)). Exemplary bioactive glasses include 45S5, 45S5B1, 58S, and 570C30. The original bioactive glass, 45S5, is melt-derived. Sol-gel derived glasses can also be produced and include nanopores that allow for increased surface area and bioactivity.

There are drawbacks to the use of bioactive glass or other materials in the form of liquids, pastes, and solids to fill voids or gaps in the bone. A liquid or a paste may not remain at the site of the void or gap in the bone. A solid may be difficult to apply and may not conform well to the void or gap in the bone. Solids may migrate or be displaced from the site through washing or other means.

These drawbacks may be reduced and/or eliminated by adding materials to a bone repair composition, such that the composition is rendered irrigation and migration resistant.

### BRIEF DESCRIPTION OF THE DRAWINGS

FIG. 1 depicts an exemplary delivery system kit for delivering an irrigation resistant bone repair composition.

FIG. 2A-B depicts schematic drawings of an adapter (2A) and a delivery gun (2B) for the irrigation resistant bone repair composition.

FIG. 3 depicts a schematic drawing of a plunger of the delivery system.

FIG. 4A depicts exemplary tips for a delivery system.

FIG. 4B depicts exemplary tips for a delivery system.

FIG. 5A is a photograph of the tubes filled with an irrigation resistant bone repair composition for use with a delivery system.

FIG. 5B depicts a schematic drawing of a tube for use with a delivery system.

FIG. 6A is a photograph of an exemplary delivery system for an irrigation resistant bone repair composition.

FIG. 6B is a photograph of an exemplary delivery system for an irrigation resistant bone repair composition.

FIG. 7 is a photograph of an exemplary delivery system for an irrigation resistant bone repair composition.

FIG. 8 is a photograph of an exemplary delivery system for an irrigation resistant bone repair composition.

FIG. 9 is a photograph of an exemplary delivery system for an irrigation resistant bone repair composition.

### SUMMARY

Certain embodiments relate to an irrigation resistant bone repair composition comprising a biocompatible bone repair material and at least one non-ionic surfactant, wherein the non-ionic surfactant is not a non-random poly(oxyalkylene) block copolymer. The non-ionic surfactant is selected from the group consisting of fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., polysorbate 20 and polysorbate 80 also referred to as Tween 20 and Tween 80), fatty acid esters or polyol esters (e.g., glycerol monostearate, PEG coconut triglycerides), polyalkylene glycols (e.g., PEG 400 and PEG 600), alkoxylated organic acids, hydroxyacids or diacids and copolymers therefrom. Preferably, one of the non-ionic surfactants in the composition has a melting point above room temperature, and more preferably above body temperature. The bone repair material can be any number of materials that assist in bone repair and production. Such materials include at least bioactive glass in the form of a particle, fiber or sphere and calcium salts, i.e., DCP, alpha TCP, beta-TCP, hydroxyapatite, calcium sulfates, calcium borates or calcium silicates, multiphasic calcium phosphates, calcium sulfates, calcium borates or calcium silicates along with elemental substitutions within these materials or coatings applied to these materials.

Further embodiments relate to bioactive glass particles including a coating comprising at least one non-ionic surfactant, wherein the non-ionic surfactant is not a non-random poly(oxyalkylene) block copolymer.

Another embodiment relates to a putty or paste including bioactive glass particles including a coating comprising at least one non-ionic surfactant, wherein the non-ionic surfactant is not a non-random poly(oxyalkylene) block copolymer.

Yet further embodiments relate to methods for treating a bone having a bone gap and/or a bone defect with the composition comprising a biocompatible bone repair material and at least one non-ionic surfactant, wherein the non-ionic surfactant is not a non-random poly(oxyalkylene) block copolymer. The non-ionic surfactant or similar material other than the non-random poly(oxyalkylene) block copolymer is selected from the group consisting of fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., polysorbate 20 and polysorbate 80), fatty alcohols, fatty acids, fatty acid esters or polyol esters (e.g., glycerol monostearate, PEG coconut triglycerides), polyalkylene glycols (e.g., PEG 400 and PEG 600), alkoxylated organic acids, hydroxyacids or diacids and copolymers therefrom. At least one of the surfactants in the composition has a melting or cloud point above room temperature, and more preferably a melting point above body temperature.

Other embodiments relate to an irrigation resistant bone repair composition comprising a biocompatible bone repair material and a mixture of non-ionic surfactants, wherein the non-ionic surfactants are not non-random poly(oxyalkylene) block copolymers. The bone repair material can be any number of materials that assist in bone repair and production. Such materials include at least bioactive glass, spherical bioactive glass in a bimodal size distribution, and tricalcium phosphate, i.e., silicated tricalcium phosphate.

Further embodiments relate to bioactive glass particles including a coating comprising two or more non-ionic surfactants, wherein the non-ionic surfactants are not non-random poly(oxyalkylene) block copolymers.

Yet another embodiment relates to a putty or paste including bioactive glass particles including a coating comprising two or more non-ionic surfactants, wherein the non-ionic surfactants are not non-random poly(oxyalkylene) block copolymers.

Yet further embodiments relate to methods for treating a bone having a bone gap and/or a bone defect with the composition comprising a biocompatible bone repair material and a mixture of non-ionic surfactants, wherein the non-ionic-surfactants are not non-random poly(oxyalkylene) block copolymers.

### DETAILED DESCRIPTION

Irrigation resistant bone repair compositions comprising (i) a biocompatible or bioactive bone repair material and at least one non-ionic surfactant, wherein the non-ionic surfactant is not a non-random poly(oxyalkylene) block copolymer, or (ii) a mixture of non-ionic surfactants, wherein the non-ionic surfactants are not non-random poly(oxyalkylene) block copolymers are provided.

Specifically, certain embodiments relate to a synthetic bone grafting composition, such as a putty for bone repair that incorporates non-ionic surfactants or other similar materials, other than a non-random poly(oxyalkylene) block copolymers, having an osteoconductive, osteostimulative and irrigation resistant properties. The term “irrigation resistant” in connection with the compositions described herein refers to a property of the composition, where the composition can be heavily irrigated following placement in a surgical site without being washed away or displaced from the surgical site. The composition includes at least one slow dissolving non-ionic surfactant(s), other than a non-random poly(oxyalkylene) block copolymers, which is mixed with a biocompatible or bioactive bone repair material, such as bioactive glasses or other osteoconductive salts, glasses or ceramics for use in methods for treating a bone having a bone gap and/or a bone defect.

The irrigation resistant bone repair composition is biocompatible and or bioactive and comprised of entirely synthetic materials, which fully eliminates any risk of disease transmission that may occur with other products containing animal or human derived materials or components to achieve this property.

The composition promotes osseointegration when introduced into a bone gap and/or a bone defect.

The bone repair composition has a unique physical property of being irrigation resistant. The irrigation resistant characteristics provide a material, which maintains position in the surgical site despite the amount of blood, body fluid or saline to which it is exposed. Irrigation resistance is beneficial to simplify the application of the bone graft at the site of defect while preventing migration of the graft material during irrigation and after closure of the surgical site. The irrigation resistance of the bone repair composition is especially beneficial for its intended use in orthopedic and spine processes, as the material will stabilize and maintain placement and structure within the body during placement, irrigation and after closure. Specifically, in certain embodiments where a non-setting putty material is used, the bone repair composition will not be displaced easily during irrigation and closure of the surgical site.

An irrigation resistant, fully synthetic and bioactive putty, when implanted into the body, will maintain position or placement rather than melt, dissolve or disintegrate during irrigation or displace upon closure of the surgical site. This feature permits the implant to hold in place more easily, and create beneficial handling properties. The ability to resist displacement allows the bioactive agent to remain at the site of implantation to stimulate bone growth for an extended period of time. The bioactive glass, as the preferred bioactive agent, stimulates the genes necessary to differentiate precursor cells into osteoblasts and the subsequent proliferation of these cells within the surgical site while undergoing an ionic exchange with the surrounding body fluid to form microcrystalline hydroxyapatite analogous to natural bone mineral. The combination of these properties in one composition is essential for bone regeneration and hard tissue repair.

The composition may be a liquid at room temperature. Alternatively, the composition may have the consistency of a solid, gel, putty, paste or any other non-liquid substance at room temperature. The composition may also have the form of a liquid, solid, gel, putty, paste or any other non-liquid substance at room temperature. Additionally the composition may undergo a phase change when warmed from room temperature to body temperature.

In some embodiments, the composition is thermoreversible changing substantially from a liquid at 5° C. and into a solid at 37° C. This effect can arise from the type and relative amount of non-ionic surfactants in the composition, which in turn determines the viscosity of the composition at room temperature and at body temperature. For example, as the temperature rises, the composition becomes substantially more viscous liquid or waxy solid to allow the bone repair material, for example, bioactive glass, to more readily remain at the defect site.

The bone repair composition provides for acceleration in the rate and an enhancement in the quality of newly-formed bone. Improved bone healing may occur in those who may be compromised, such as diabetics, smokers, the obese, the elderly, those who have osteoporosis, those who use steroids, and those who have infections or other diseases that reduce the rate of healing. The rapid hardening of the bone repair composition at the site of the bone defect can serve to localize the bone repair material, such as bioactive glass, at the site.

The bone repair composition may be provided to a site of a bone defect by means of a syringe or other injection device. In certain embodiments, the bone repair composition may be sufficiently liquid so as to be injectable, yet can harden suitably at the bone defect site at body temperature. For instance, if the bone repair composition is a liquid at room temperature, it may become a thick gel at body temperature; in other words, the bone repair composition cures upon application to a bone defect at body temperature.

In certain embodiments, the bone repair composition has the advantages of low viscosity, runny liquid composition with regard to the ease of application to a bone defect site. Further advantages of the composition include more solid paste-like composition characteristics and that it remains positioned at the defect after being applied. The solidification of the composition at body temperature overcomes the disadvantageous property of other liquid compositions that do not exhibit irrigation resistant behavior. At the same time, because the composition is not a solid at room temperature, there is greater ease of applying the composition, such as by means of a syringe. The composition need not be laboriously painted onto a bone defect or applied onto a bone defect by means of pressure.

Other delivery modes can be used for more viscous bone repair compositions. These modes include manually placing the gel or paste directly into a bone defect or extruding the gel or paste using a syringe, delivery gun or other means.

In certain embodiments, if the bone repair composition is a gel at room temperature, it may become a paste at body temperature.

In certain other embodiments, if the bone repair composition is a thick gel or paste at room temperature, it may become putty or a solid at body temperature.

As noted above, the relative amount of a non-ionic surfactant (other than a non-random poly(oxyalkylene) block copolymers), in the composition will determine the viscosity at room temperature and at body temperature.

In certain embodiments, the irrigation resistant composition includes a biocompatible or bioactive bone repair material, and at least one non-ionic surfactant, with the proviso that the non-ionic surfactant is not a non-random poly(oxyalkylene) block copolymer.

The non-ionic surfactant or similar material other than the non-random poly(oxyalkylene) block copolymer is selected from the group consisting of fatty acids (e.g. stearic acid), fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 monostearate), alkoxylated fatty ethers (e.g., polyethylene glycol lauryl ether (Brij L23), polyglycerin fatty acid esters, alkoxylated sorbitan esters (e.g., Span 85 (sorbitan trioleate)), alkoxylated sorbitan esters (e.g., Polysorbate 20 and Polysorbate 80 also referred to as Tween 20 and Tween 80), fatty acid esters or polyol esters (e.g., glycerol monostearate, PEG coconut triglycerides), polyalkylene glycols (e.g., PEG 400 and PEG 600), alkoxylated organic acids, hydroxyacids or diacids and copolymers therefrom. Specific examples of non-ionic surfactants, other than the non-random poly(oxyalkylene) block copolymers, include sorbitan tristearate, polysorbate 20, polysorbate 80, polyoxyethylene 7 coconut, glycerides, poly(ethylene glycol) 400 monostearate (PEG 400 monostearate), PEG 2000 monomethylether, and PEG 400 distearate.

Further examples of the non-ionic surfactants suitable for use with the irrigation resistant compositions include polyglyceryl-2 isostearate, polyglyceryl-2 diisostearate, polyglyceryl-4 isostearate, polyglyceryl-6 isostearate, poly(ethylene glycol) 8 stearate (MYRJ S8), polyglyceryl-10 isostearate, polyglyceryl-10 diisostearate, poly(ethylene glycol) 25 propylene glycol stearate (MYRJ S25), poly(ethylene glycol) 400 distearate (PEG 400 distearate), polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, and polyglyceryl-10 distearate.

Yet further examples of the non-ionic surfactants include polyoxyethylene 7 coconut glyceride (coconut glyceride), polyethylene glycol 2000 monomethyl ether (MME), glyceryl monostearate (monostearin), PEG dimethyl ether (dimethyl polyethylene glycol), PEG 200 adipate (poly(ethylene glycol) 200 adipate, PEG 6000 distearate, sorbitan monostearate, cetyl alcohol, ethylene glycol monostearate, propylene glycol stearate, polyoxyethylene stearyl ether (Brij 2), polyoxyethylene stearyl fatty ether (Brij 10), docosaethylene glycol mono octadecyl ether (Brij 20), polyethylene stearyl ether (Brij 100), polyglycerin fatty acid ester (polyglyceryl-2 isostearate, polyglyceryl-2 diisostearate, polyglyceryl-4 isostearate, polyglyceryl-6 isostearate, polyglyceryl-10 isostearate, polyglyceryl-10 diisostearate, polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, polyglyceryl-10 distearate).

In certain embodiments, one of the surfactants in the composition has a melting point or cloud point above room temperature, and more preferably above a melting point above body temperature.

In certain other embodiments, at least two non-ionic surfactants, other than a non-random poly(oxyalkylene) block copolymers, may be included; alternatively, at least three or more non-ionic surfactants, other than a non-random poly(oxyalkylene) block copolymers, may be included.

In some embodiments, the weight ratio of at least one non-ionic surfactant, other than poly(oxyalkylene) block copolymer is 1%-99% relative to the weight of the bone repair composition. This weight ratio may be from 1-10%, 10-20%, 20-30%, 30%-40%, 40%-50%, 50%-60%, 60%-70%, 70%-80%, 80%-90%, or 90-99%. Alternatively, this weight ratio may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. The material may have the consistency of a solid, gel, putty, paste or any other non-liquid substance at room temperature.

In some embodiments, the weight ratio of the mixture of at least two non-ionic surfactant, other than a non-random poly(oxyalkylene) block copolymers, is 1%-99% relative to the weight of the bone repair composition. This weight ratio may be from 1-10%, 10-20%, 20-30%, 30%-40%, 40%-50%, 50%-60%, 60%-70%, 70%-80%, 80%-90%, or 90-99%. Alternatively, this weight ratio may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. The material may have the consistency of a solid, gel, putty, paste or any other non-liquid substance at room temperature.

In some embodiments, where the bone repair composition comprises two non-ionic surfactants, the weight ratio of a first non-ionic surfactant to the weight ratio of a second non-ionic surfactant is in the range of from about 1%-99% to about 99%-1%. Specifically, the weight ratio of a first non-ionic surfactant to the weight ratio of a second non-ionic surfactant is about 1% to 99%; alternatively, the weight ratio of a first non-ionic surfactant to the weight ratio of a second non-ionic surfactant is about 50% to 50%; and alternatively, the weight ratio of a first non-ionic surfactant to the weight ratio of a second non-ionic surfactant is about 99% to 1%. The compositions may vary in molecular weight and be blended in ratios of 10:1 to 1:10.

The compositions may further comprise ions and other compounds that may be dissolved in water. For example, the addition of salts, such as PBS, can enhance solidification and setting properties of non-ionic surfactants. Divalent salts may be particularly useful to improve the rheological properties of compositions containing non-ionic surfactant mixtures and bioactive glass materials as well as those of compositions containing non-ionic surfactants and other solid bone repair materials.

In certain embodiments, the composition may further include at least one additive, including but not limited to solvents, linear, straight chain and branched aliphatic hydrocarbons, sugars, polysaccharides, and hydroxyl or alkoxy terminal polyalkylene oxides along with low molecular weight biodegradable polymers (MW</=10,000).

Specific examples of additives include sodium hyaluronate, regenerez, polypropylene glycol 3000 (poly 3000), seasame oil, candelilla wax, carnauba wax, sorbitol (D-Glucitol), polycaprolactone, polycaprolactone diol, coconut oil, propylene glycol, polycaprolactone triol, polycaprolactone 10000 mw, mineral oil high viscosity, mineral oil low viscosity, polyethylene glycol 400 (PEG-8), butylene glycol, and hexylene glycol.

The biocompatible or bioactive bone repair material may be osteoinductive, osteoconductive, or a material that is both osteoinductive and osteoconductive. The bone repair material may be xenogeneic, allogeneic, autogeneic, and/or allo-plastic.

The bone repair material can be any number of materials that assist in bone repair and production. Such materials include at least bioactive glass in the form of a particle, sphere, fiber, mesh, sheet or a combination of these forms, i.e. fibers within a sphere, and calcium salts, i.e., DCP, alpha TCP, beta-TCP, hydroxyapatite, calcium sulfates, calcium borates or calcium silicates, multiphasic calcium phosphates, calcium sulfates, calcium borates or calcium silicates along with elemental substitutions within these materials or coatings applied to these materials. In certain embodiments, the biocompatible or bioactive bone repair material may also be any combination of various therapeutic materials. The various types of bioactive glass that may be used as bone repair material were previously described In U.S. Pub. No. US 2014/0079789, entire content of which is incorporated herein by reference.

In certain embodiments, the composition may be prepared as a composite with a biocompatible or bioactive agent, such as a bioactive glass ceramic which contains silica or boron. The ceramic releases calcium and silicate or calcium and boron ions, which facilitate the differentiation and proliferation of osteoblasts (defined as osteostimulation), which in turn increases the rate of regeneration of hard tissue.

In addition, the bioactive glass component undergoes an ion exchange with the surrounding body fluid to form hydroxyapatite analogous to bone mineral. More specifically, dissolution of the bioactive glass ceramics releases the calcium and silicate or calcium and boron ions, which stimulate the genes responsible of the differentiation and proliferation of osteoblast cells within the bony defect upon implantation. It is believed that this genetic response is activated through the introduction of the genetic cascade responsible for the osteoblast proliferation and subsequently promotes the increased rate of regeneration of hard tissue.

In certain embodiments, the bone repair material is bioactive glass. Bioactive glass may be melt-derived or sol-gel derived. Depending on their composition, bioactive glasses may bind to soft tissues, hard tissues, or both soft and hard tissues. The composition of the bioactive glass may be adjusted to modulate the degree of bioactivity. Furthermore, borate may be added to bioactive glass to control the rate of degradation.

In some embodiments, the bioactive glass contains silica and/or boron as well as other ions such as sodium and calcium.

Certain embodiments relate to an irrigation resistant bone repair composition that includes a biocompatible or bioactive bone repair material suspended in a mixture of at least two non-ionic surfactant, other than a non-random poly(oxyalkylene) block copolymer.

Certain further embodiments relate to an irrigation resistant bone repair composition that further includes at least one element selected from the group consisting of Li, Na, K, Mg, Sr, Ti, Zr, Fe, Co, Cu, Zn, Al, Ga, P, N, S, F, CI, and I. For example, small amounts of iodine, fluorine or silver can provide antimicrobial properties, while small amount of copper can promote angiogenesis (i.e., aid in the formation of blood vessels).

The preferred embodiment includes non-ionic surfactants, other than a non-random poly(oxyalkylene) block copolymers, as carriers for melt and sol-gel derived bioactive glasses. The composites range from 1 to 99% of a mixture of non-ionic surfactants, other than a non-random poly(oxyalkylene) block copolymers, which is conversely 1-99% bioactive glass.

The bioactive glass may be in a form of particles, spheres, fibers, mesh, sheets or a combination of these forms i.e. fibers within a sphere.

The compositions may vary in molecular weight and may be blended in ratios of 10:1 up to 1:10. Compositions of the glass may comprise from 0-90% silica or 0-90% boric acid with a plurality of other elements including Li, Na, K, Mg, Sr, Ti, Zr, Fe, Co, Cu, Zn, Al, Ga, P, N, S, F, CI, and I. The embodiments take the consistency of a gel, putty, or waxy solid at room temperature.

In certain embodiments, bioactive glass is in the form of a particle. The composition, porosity and particle sizes of the bioactive glass may vary. In certain preferred embodiments, the particles of the glass may range in size from 0.01 μm to 5 mm. In certain embodiments, the bioactive glass comprises 0-80%<100 μm, 0-80%<500 μm, 0-80% 500-1000 μm, 0-80% 1000-2000 μm, 0-80% 2000-5000 μm, 0-90% 90-710 μm, and 0-90% 32-125 μm bioactive glass.

Specifically, the bioactive glass material may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the corn-position in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, and boron with only traces of the other(s). The term “about” as it relates to the amount of calcium phosphate present in the composition means+/−0.5%. Thus, about 5% means 5+/−0.5%.

The bioactive glass materials may further comprise one or more of a silicate, horosilicate, borate, strontium, or calcium, including SrO, CaO, P₂O₅, SiO₂, and B₂O₃. In certain embodiments, bioactive glass includes about 15-45% CaO, about 30-70% SiO₂, about 0-25% Na₂O, about 0-17% P₂O₅, about 0-10% MgO and about 0-5% Ca F₂.

An exemplary bioactive glass is 45S5, which includes 46.1 mol % SiO₂, 26.9 mol % CaO, 24.4 mol % Na₂O and 2.5 mol % P₂O₅.

An exemplary borate bioactive glass is 45S5B1, in which the SiO₂ of 4535 bioactive glass is replaced by B₂O₃.

Other exemplary bioactive glasses include 58S, which includes 60 mol % SiO₂, 36 mol % CaO and 4 mol % P₂O₅, and S70C30, which includes 70 mol % SiO₂ and 30 mol % CaO.

In any of these or other bioactive glass materials of the invention, SrO may be substituted for CaO.

The following composition provided in Table 1 below, having a weight % of each element in oxide form in the range indicated, will provide one of several bioactive glass compositions that may be used to form a bioactive glass material:

TABLE 1

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

In case of the bioactive glass being in the form of a three-dimensional compressible body of loose glass-based fibers, the fibers comprise one or more glass-formers selected from the group consisting of P₂O₅, SiO₂, and B₂O₃. Some of the fibers have a diameter between about 100 nm and about 10,000 nm, and a length:width aspect ratio of at least about 10. The pH of the bioactive glass can be adjusted as-needed.

The bioactive glass particles, fibers, spheres, meshes or sheets may further comprise any one or more of adhesives, grafted bone tissue, in vitro-generated bone tissue, collagen, calcium phosphate, stabilizers, antibiotics, antibacterial agents, antimicrobials, drugs, pigments, X-ray contrast media, fillers, and other materials that facilitate grafting of bioactive glass to bone.

The silica and/or calcium ions released by the bioactive glass may improve the expression of osteostimulative genes. The silica and/or calcium ions may also increase the amount of and efficacy of proteins associated with such osteostimulative genes. In several embodiments, the bone repair material is osteostimulative and can bring about critical ion concentrations for the repair and regeneration of hard tissue without the necessity of any therapeutic materials or agents.

In some embodiments, the bone repair material is 45S5 bioactive glass. The 45S5 bioactive glass may vary in size from 1 micrometer to 5 millimeters. The bioactive glass may be about 1-5 micrometers, about 5-15 micrometers, about 15-50 micrometers, about 50-200 micrometers, about 200-1,000 micrometers, about 1-2 millimeters, about 2-3 millimeters, about 3-4 millimeters, or about 4-5 millimeters.

In some embodiments, the bioactive glass particle has a diameter of between about 0.01 micrometer and about 5,000 micrometers.

In some embodiments, the bone repair material is a composition comprising calcium salt and silica. The silica is in the form of an inorganic silicate that is ad-sorbed onto the surface of the calcium salt. The silica is not incorporated into the structure of the calcium salt. The composition may be bioactive. These and other bone repair materials are described in U.S. Patent Pub. No. US 2013/0330410, the entire content of which is herein incorporated by reference.

In some embodiments, the bone repair material is a composition comprising suspended autograft bone particles and suspended bioactive glass particles. Similar bone repair materials are described in U.S. Pub. No. 2015/0079146, the entire content of which is incorporated by reference.

The suspended bioactive glass particle may comprise SiO₂. Alternatively, the suspended bioactive glass particle may comprise P₂O₅, PO₃. or PO₄. The suspended bioactive glass particle may comprise B₂O₃ as well. In some embodiments, the suspended bioactive glass particle may comprise 40-60% SiO₂, 10-20% CaO, 0-4% P₂O₅, and 19-30% NaO. The suspended bioactive glass particle may further comprise a carrier selected from the group consisting of hydroxyapatite and tricalcium phosphate.

The bioactive glass particles, fibers, meshes or sheets may be pretreated in a solution comprising one or more of blood, bone marrow aspirate, bone-morphogenetic proteins, platelet-rich plasma, and osteogenic proteins.

In some embodiments, the bone repair material may be bioactive glass coated with a glycosaminoglycan, in which the glycosaminoglycan is bound to the bioactive glass. This and other bone repair materials are described in U.S. Patent Pub. No. US 2014/0079789, the entire content of which is incorporated by reference. The glycosaminoglycan may be bound to the bioactive glass by means of an ionic bond or a covalent bond. The glycosaminoglycan may be heparin, heparan sulfate, chondroitin sulfate, dermatan sulfate, keratan sulfate, or hyaluronic acid.

In certain other embodiments, the bone repair material may include surface immobilized peptides, as previously described in U.S. patent application Ser. No. 14/504,956, filed on Oct. 2, 2014, which is incorporated herein in its entirety.

In some further embodiments, the bone repair material is a bimodal bioactive glass composition comprising large bioactive glass particles and small bioactive glass particles. The large bioactive glass particles have a substantially spherical shape and a mean diameter of between about 90 micrometers and about 2,000 micrometers. The small bioactive glass particles have a substantially spherical shape and a mean diameter of between about 10 micrometers and about 500 micrometers.

In some embodiments, the bone repair material is a trimodal bioactive glass composition comprising large bioactive glass particles, medium bioactive glass particles, and small bioactive glass particles. The large bioactive glass particles have a substantially spherical shape and a mean diameter of between about 500 micrometers and about 5,000 micrometers. The medium bioactive glass particles have a substantially spherical shape and a mean diameter of between about 90 micrometers and about 710 micrometers. The small bioactive glass particles have a substantially spherical shape and a mean diameter of between about 1 micrometers and about 125 micrometers.

In any of the above embodiments, small bioactive glass fibers may be added to the bone repair material. The small bioactive glass fibers have a diameter of less than 2 millimeters. The small bioactive glass fibers may be present in up to 40% by weight relative to the total weight of the bioactive glass. In various embodiments, the weight ratio of small bioactive glass fibers to total weight of the bioactive glass may be from 0-10%, 0-5%, 5-10%, 5-15%, 10-15%, 10-20%, 15-20%, 15-25%, 20-25%, 20-30%, 25-30%, 25-35%, 30-35%, 30-40%, or 35-40%. The weight ratio of small bioactive glass fibers to total weight of the bioactive glass may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, any subset of the bioactive glass present, such as bioactive glass particles and/or small bioactive glass fibers, may be coated with silane as described in Verne et al. (Verne et al., “Surface functionalization of bioactive glasses,” J. Biomed. Mater. Res. A., 90(4):981-92 (2009)). The silane or other functional coatings may then allow for binding of proteins onto the bioactive glass, such as BMP-2.

In some embodiments, any subset of the bioactive glass present, such as bioactive glass particles and/or small bioactive glass fibers, may have additional silicate chains present on them. The additional silicate chains may allow the bioactive glass particles and fibers to interact with one another, as well as with groups of the non-ionic surfactants. The effect of these interactions may be to reduce the surface area of the filler, increase resin demand, and to allow for higher filler loadings.

In some embodiments, any subset of the bioactive glass present, such as bioactive glass particles and/or small bioactive glass fibers, may have added hydroxyl triethoxysilanes coated onto the glass. Some of these silanes are available from Gelest, Inc. For example, the glass may be coated with hydroxyl(polyethyleneoxy) propyltriethoxysilane. Additionally, the glass may be coated with other organic substituted ethoxy- and methoxy-silanes that are effective to create an interaction between the coated glass and the EO/PO carrier.

In any of the above embodiments, the irrigation resistant bone repair composition may be applied by a syringe at ambient temperature. After application to the bone or other site within the body at 37° C., the bone repair composition will harden and have a substantially lower tendency to migrate away from the application site.

More viscous bone repair compositions may be applied by painting the composition onto a site at or near the bone defect. Alternatively, more viscous bone repair compositions may be extruded onto the site in the form of a bead.

Certain embodiments relate to a method for treating hard tissues, such as bones using the irrigation resistant bone repair composition.

Certain other embodiments relate to a method for treating a bone having a bone defect comprising contacting the bone at or near the site of the bone defect with the irrigation resistant bone repair composition of any of the above-described embodiments.

Any of the above-described materials or methods may be undertaken to treat any number of bone defects. As such, certain further embodiments relate to a method for treating a bone having a bone defect comprising placing an irrigation resistant bone repair composition of any one of the above-described embodiments at a site of a bone gap or a bone defect.

A bone defect may include bony structural disruptions, in which repair is needed or may be a gap in the bone or may arise from lack of adequate bone regeneration. A bone defect may be a void, which is understood to be a three-dimension defect that includes a gap, cavity, hole or other substantial disruption of the structural integrity of the bone or joint. The bone defects may also be fractures. The bone defects may also arise in the context of oral bone defects. The different types of bone defects are apparent to those of ordinary skill in the art. Gaps may be at least 2.5 cm and are generally in the range of 3-4 cm. This size is large enough so that spontaneous repair is not likely to occur and/or be complete. Exemplary bone defects include tumor resection, fresh fractures, cranial and facial abnormalities, spinal fusions, and loss of bone from the pelvis.

The various embodiments of the invention may be particularly useful with respect to orthopedic and spine processes because the material will stabilize and hold a better structure as it becomes more solidified when it heats up to body temperature.

Certain further embodiments relate to a method for treating a bone having a bone defect comprising placing an irrigation resistant bone repair composition of any one of the above-described embodiments at a bone gap or a bone defect.

In some embodiments, any of the above-described materials or methods may be combined with autograft bone chips for placement onto or near a bone defect. The materials may be a liquid or a gel at room temperature with the autograft bone chips suspended therein. Upon placement at or near the bone defect, the material will solidify around the autograft bone chips and serve to prevent the autograft bone chips from migrating away from the surgical sites.

In some embodiments, any of the above-described materials or methods may be combined with particles containing allogeneic or xenogeneic bone mineral for placement onto or near a bone defect. The materials may be a liquid or a gel at room temperature with the particles suspended therein. Upon placement at a surgical site, which is at or near the bone defect, the material will solidify around the particles and serve to prevent the particles from migrating away from the surgical site.

In various embodiments of the invention, the bone repair material is not a natural bone material or a synthetic bone material.

Further embodiments relate to kits that include an irrigation resistant bone repair composition including a biocompatible or bioactive bone repair material, and at least one surfactant other than the non-random poly(oxyalkylene) block copolymer. The non-ionic surfactant or similar material, other than the non-random poly(oxyalkylene) block copolymer, is selected from the group consisting of fatty acids (e.g. stearic acid), fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 Monostearate), alkoxylated fatty ethers (e.g., polyethylene glycol lauryl ether (Brij L23), polyglycerin fatty acid esters, alkoxylated sorbitan esters (e.g., Span 85 (sorbitan trioleate)), alkoxylated sorbitan esters (e.g., Polysorbate 20 and Polysorbate 80 also referred to as Tween 20 and Tween 80), fatty acid esters or polyol esters (e.g., glycerol monostearate, PEG coconut triglycerides), polyalkylene glycols (e.g., PEG 400 and PEG 600), alkoxylated organic acids, hydroxyacids or diacids and copolymers therefrom. Specific examples of non-ionic surfactants, other than the non-random poly(oxyalkylene) block copolymers, include sorbitan tristearate, polysorbate 20, polysorbate 80, polyoxyethylene 7 coconut, glycerides, poly(ethylene glycol) 400 monostearate (PEG 400 monostearate), PEG 2000 monomethylether, and PEG 400 distearate. Further examples of the non-ionic surfactants suitable for use with the irrigation resistant compositions include polyglyceryl-2 isostearate, polyglyceryl-2 diisostearate, polyglyceryl-4 isostearate, polyglyceryl-6 isostearate, poly(ethylene glycol) 8 stearate (MYRJ S8), polyglyceryl-10 isostearate, polyglyceryl-10 diisostearate, poly(ethylene glycol) 25 propylene glycol stearate (MYRJ S25), poly(ethylene glycol) 400 distearate (PEG 400 distearate), polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, and polyglyceryl-10 distearate. Yet further examples of the non-ionic surfactants include, polyoxyethylene 7 coconut glyceride (coconut glyceride), polyethylene glycol 2000 monomethyl ether (MME), glyceryl monostearate (monostearin), PEG dimethyl ether (dimethyl polyethylene glycol), PEG 200 adipate (poly(ethylene glycol) 200 adipate, PEG 6000 distearate, sorbitan monostearate, cetyl alcohol, ethylene glycol monostearate, propylene glycol stearate, polyoxyethylene stearyl ether (Brij 2), polyoxyethylene stearyl fatty ether (Brij 10), docosaethylene glycol mono octadecyl ether (Brij 20), polyethylene stearyl ether (Brij 100), polyglycerin fatty acid ester (polyglyceryl-2 isostearate, polyglyceryl-2 diisostearate, polyglyceryl-4 isostearate, polyglyceryl-6 isostearate, polyglyceryl-10 isostearate, polyglyceryl-10 diisostearate, polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, polyglyceryl-10 distearate).

Further embodiments relate to kits that include an irrigation resistant bone repair composition including a biocompatible or bioactive bone repair material, and a mixture of at least two non-ionic surfactants or similar materials.

Exemplary kits for use with bone resistant compositions were previously described in U.S. Pub. No. US 2015/030684, which is incorporated herein in its entirety.

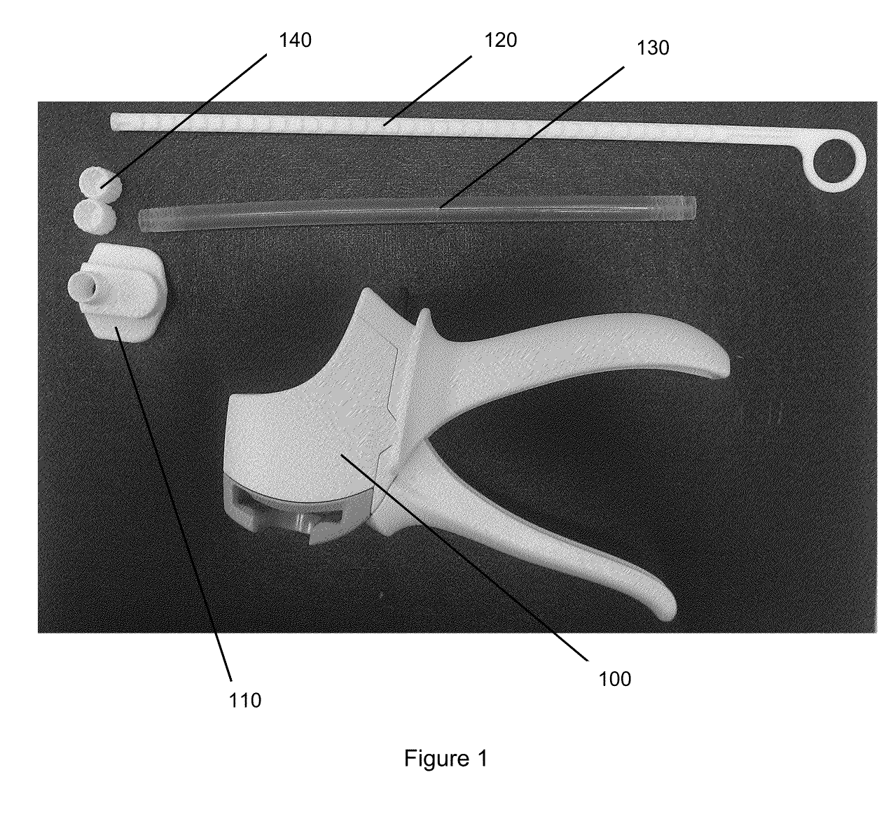

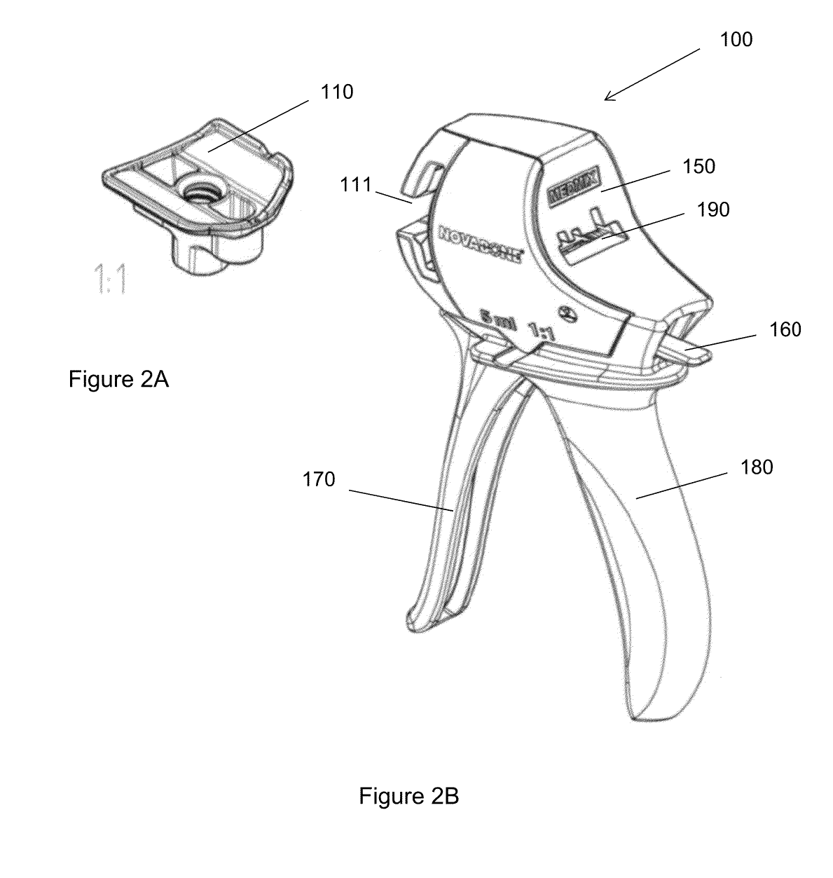

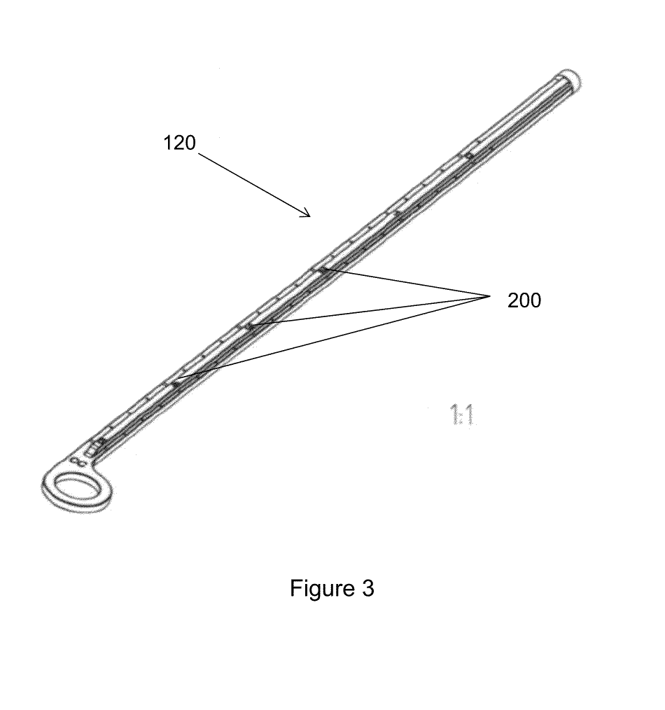

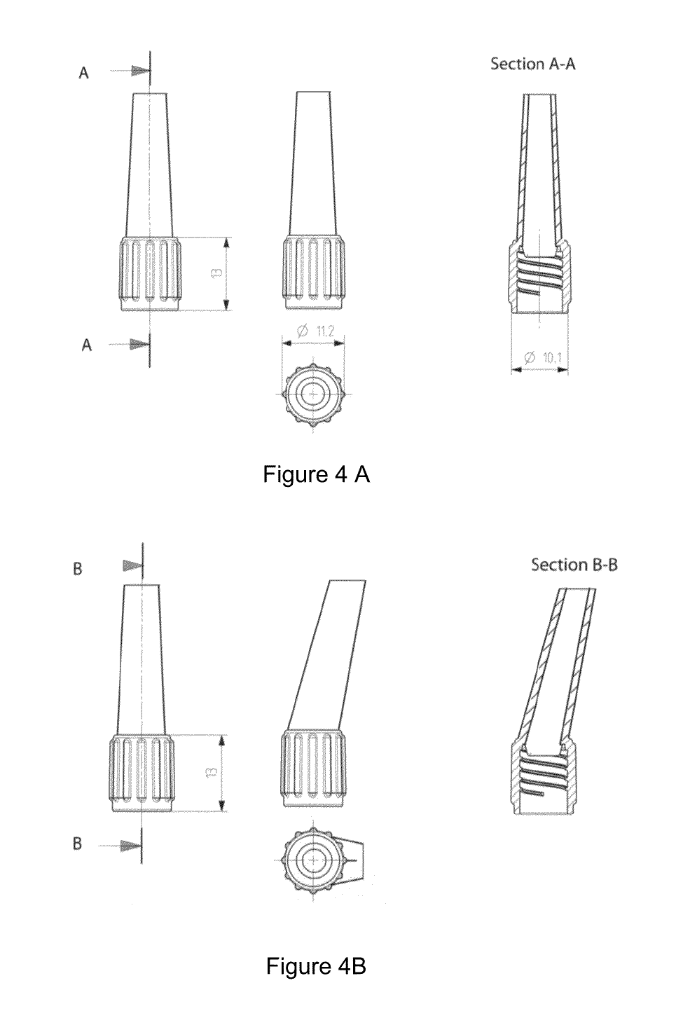

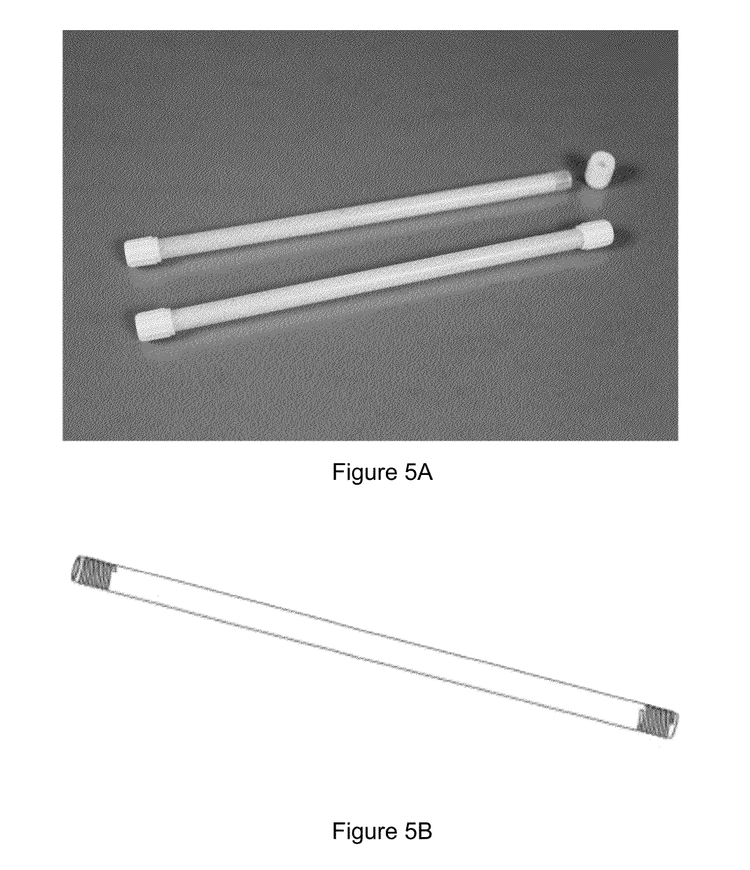

The kits may further include a dispensing gun, syringe, clam shell, or other suitable delivery device and accompanying accessories. Specifically, referring to FIGS. 1 and 2A-B, the exemplary dispensing gun 100, adapter 110, plunger 120 (see also FIG. 3), tube(s) 130 (see also FIGS. 5A and 5B), caps 140, and assorted dispensing tips (optional; FIG. 4A and FIG. 4B) that may be included with the kits are shown. The irrigation resistant bone repair composition may be deposited into the tube(s) 130 as part of the kit (FIG. 5A). An exemplary kit for delivery of other materials, such as Bioactive Synthetic Bone Graft Putty is currently being sold by NOVABONE® (NOVABONE® Bioactive Synthetic Bone Graft Putty MIS Cartridge Delivery System, NovaBone Products, LLC, Alachua, Fla.).

Referring to FIGS. 2A-B, the dispensing gun 100 may include a cover 150, a latch 160, a lever 170 and a handle 180 (FIG. 2B). The adapter 110 (shown also in FIG. 2A) may be inserted into the dispensing gun at an opening 111. A plunger (not shown) may be inserted through the front of the gun and pushed through the opening in the back 190 of the gun.

FIG. 3 depicts an exemplary plunger 120 including gradient markings 200 facing up.

FIGS. 4A-B depict exemplary tips for use with the dispensing gun. The tips may be straight (FIG. 4A) or at an angle (FIG. 4B).

FIG. 5A is a picture of tubes filled with the irrigation resistant bone repair composition; FIG. 5B is a graphical illustration of an exemplary tube for use with the kit and specifically with the delivery gun described above. The tubes have a substantially constant inner diameter along their entire length such that the outlets have substantially the same inner diameters as the rest of the tubes.

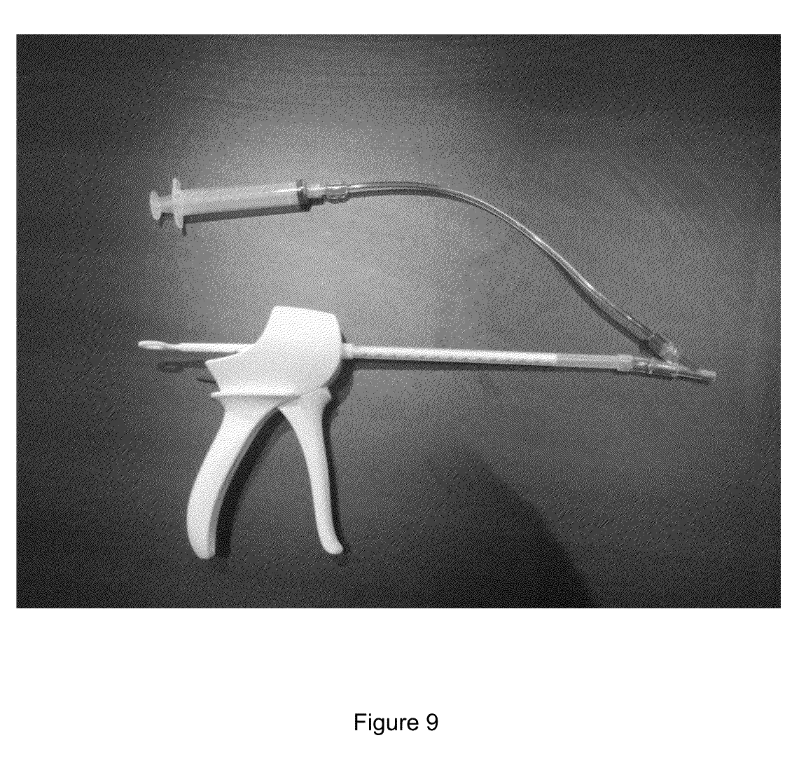

Optionally, a “Y” connector, luer syringe and a tube connector may be included to facilitate the simultaneous delivery of biologics and to maintain position during shipping (as shown in FIG. 9).

The components of a kit may be packaged and sold as a kit. The components of a kit may snap fit into a (inner) tray of a packaging and a retainer may be placed over the components of the kit to maintain position of the components during shipping. The inner tray may hold up to four tubes that can be prefilled with the irrigation resistant bone repair composition and capped on each end. The inner tray may also contain cavities for the placement of assorted tips, a “Y” connector, tube connector, a syringe and aspiration needle.

The inner tray may be sealed with a lid and placed into an outer tray also sealed with a lid. The sealed trays are radiation sterilized for use in medical applications. The sealed trays may then be placed in a box.

Immediately prior to use, the kit may be placed in an operating room and the outer tray is opened. The inner tray is removed by a sterile technician and placed into the sterile field.

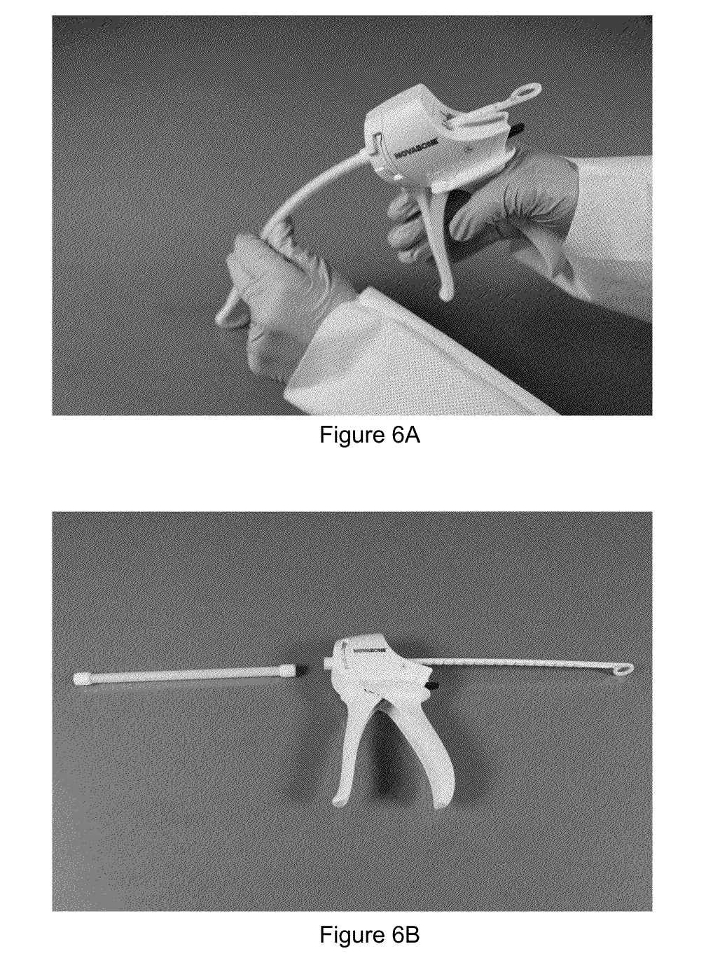

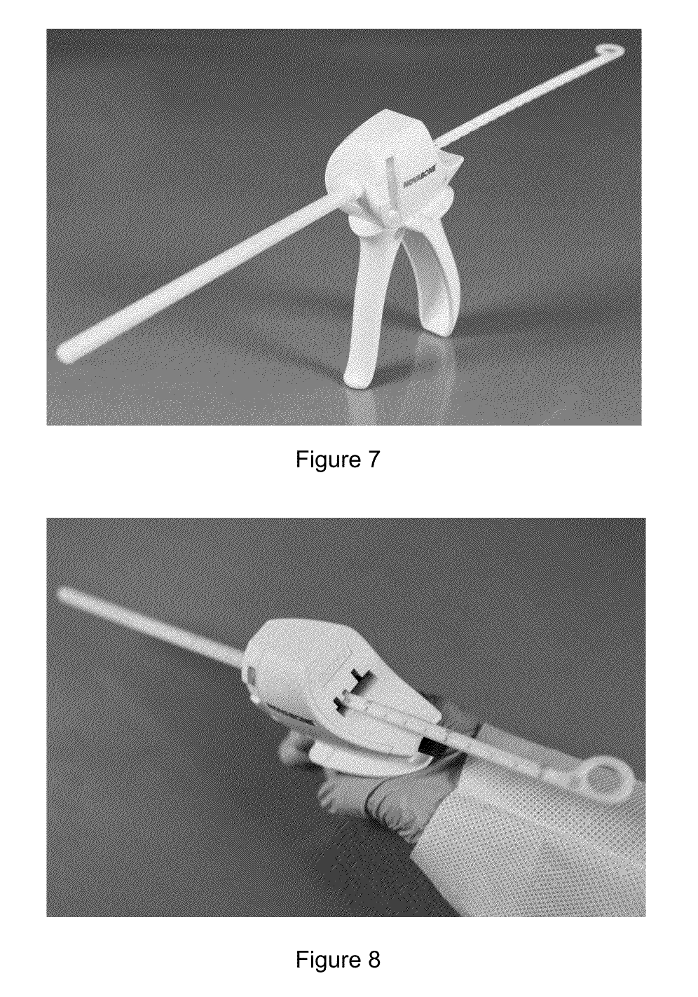

In the sterile field the inner tray is opened and the dispensing gun is assembled by inserting the finger grip of the plunger 120 (with the gradient markings 200 facing up and teeth facing down) through the opening in the front of the gun 100 and pushing the plunger through the back of the gun until the piston end of the plunger is seated completely within the gun (see FIGS. 6A, 7 and 8). The adapter 110 is then inserted into the front of the gun 100. Next a prefilled tube is removed from the inner tray. One cap is removed from the prefilled tube. The tube is threaded into the adapter and the other cap is removed from the tube (FIG. 6B). Optionally a tip can be placed on the end of the tube to direct the flow of the graft material.

The tip of the instrument may be placed into the surgical site. Upon pressing the trigger of the gun, the plunger is ratcheted forward to express the bone grafting material into the surgical site. The dispensing gun consists of, a handle, in which a block is moved forward through pressing the trigger which engages the teeth of the plunger moving the piston forward displacing the material from the tube. The trigger is manually disengaged by pushing the lever at the back of the dispensing gun upward allowing the plunger to be pulled back to a starting position. The first tube can be removed from the adapter and additional tubes can be threaded in place as needed.

Another embodiment involves altering the adapter for the attachment of two tubes and the plunger modified from a single piston to one have two pistons moving simultaneously with each compression of the trigger. Subsequently, the plungers dispense the material from the two tubes through a static mixer to facilitate the addition of a biological or drug material into the non-setting bone grafting material during injection into the surgical site. Any of the above-described aspects and embodiments of the invention may be in injectable form. Injection may occur by means of a syringe, for example. The compositions are particularly useful when injected in a gel or liquid form into a bone gap or bone defect. The injected gel or liquid would then solidify at body temperature when placed on or near the bone gap or the bone defect.

### Example 1

The purpose of the study was to establish a standard for evaluating the handling of irrigation resistant matrix (IRM) samples when immersed in water.

Materials used in the study were IRM samples as noted in Tables 2-28, deionized water, and crystallization dish. Equipment used included hotplate and analytical balance.

To preparing for the test deionized water was dispensed into the crystallization dish so that a 5-7.5 g IRM sample was completely immersed. The crystallization dish was then set on the hot plate and heated to 37° C.

Next to run the immersed compression test, each IRM sample (5-7.5 g) was molded into a sphere. With the crystallization dish still on the hotplate, each sample was pressed flat against the bottom of the dish. The sample was then picked up out of the water bath, remolded into a sphere, and the process was repeated. The steps were repeated until the sample could no longer be picked up or no longer molded into a sphere.

The pass/fail criteria included: each sample that was pressed and picked up more than once passed the testing; each sample that was only pressed and picked up once failed the testing. The term “immersed compression” refers to the property of stiffness or the ability of the material to be manipulated under warm water while the material is still intact.

The grading used included: 0 and 1 unsatisfactory; 2-4 acceptable; and >4 outstanding.

The results are shown in Tables 2-28.

TABLE 2

| | % | Total | | BG | | MYRJ | Sodium | Polyglycerol | Immersed |
|---|---|---|---|---|---|---|---|---|---|
| | BG | mass | BG 1-2 mm | 90 μm-710 μm | BG 32 μm-125 μm | S25 | Hyaluronate | sebacate | Compression |
| % | 67 | | 30.15 | 26.80 | 10.05 | 13.2 | 0.3 | 19.8 | 0 |
| Kg-01- | 67 | 50 | 15.075 | 13.4 | 5.025 | 6.6 | 0.15 | 9.9 | 0 |
| 08-B-1 | | | | | | | | | |

TABLE 3

| | | | All | | | PEG | PEG | Poly- | Glyceryl | | |
|---|---|---|---|---|---|---|---|---|---|---|---|
| | % | Total | bio- | BG | BG | 400 | 400 di- | propylene | Mono- | Sodium | Immersed |
| | BG | mass | glass | 90 μm-710 μm | 32 μm-125 μm | monostearate | stearate | glycol | stearate | Hyaluronate | Compression |
| % | 73 | | 0.00 | 56.00 | 13.00 | 5.7 | 14.5 | 5.7 | 5.7 | 0.3 | |
| Kg-01- | 73 | 47.6 | 0 | 28 | 6.5 | 7.25 | 2.85 | 0 | 2.85 | 0.15 | 0 |
| 18-A | | | | | | | | | | | |
| Kg-01- | 73 | 47.6 | 0 | 28 | 6.5 | 2.85 | 8.25 | 0 | 1.85 | 0.15 | 0 |
| 18-B | | | | | | | | | | | |
| Kg-01- | 73 | 47.6 | 0 | 28 | 6.5 | 2.85 | 7.25 | 0 | 2.85 | 0.15 | 0 |
| 18-C | | | | | | | | | | | |
| Kg-01- | 73 | 47.6 | 0 | 28 | 6.5 | 0 | 7.25 | 2.85 | 2.85 | 0.15 | 0 |
| 18-D | | | | | | | | | | | |
| Kg-01- | 73 | 47.6 | 0 | 28 | 6.5 | 0 | 7.75 | 3.35 | 1.85 | 0.15 | 0 |
| 18-E | | | | | | | | | | | |

TABLE 4

| | | | | | PEG | PEG | Poly- | Glyceryl | | |
|---|---|---|---|---|---|---|---|---|---|---|
| | % | Total | BG | BG | 400 | 400 | propylene | Mono- | Sodium | Immersed |
| | BG | mass | 90 μm-710 μm | 32 μm-125 μm | MONO | distearate | glycol | stearate | Hyaluronate | Compression |
| % | 73 | | 56.00 | 13.00 | 5.7 | 14.5 | 5.7 | 5.7 | 0.3 | |
| Kg-01- | 73 | 95.2 | 56 | 13 | 14.5 | 5.7 | 0 | 5.7 | 0.3 | 6 |
| 19-A1 | | | | | | | | | | |
| Kg-01- | 73 | 95.2 | 56 | 13 | 14.5 | 0 | 5.7 | 5.7 | 0.3 | 4 |
| 19-A2 | | | | | | | | | | |
| Kg-01- | 73 | 95.2 | 56 | 13 | 0 | 15.5 | 6.7 | 3.7 | 0.3 | 3 |
| 19-E1 | | | | | | | | | | |
| Kg-01- | 73 | 95.2 | 56 | 13 | 0 | 15.5 | 6.7 | 3.7 | 0.3 | 4 |
| 19-E2 | | | | | | | | | | |

TABLE 5

| | | | | | PEG | PEG | | | | | |
|---|---|---|---|---|---|---|---|---|---|---|---|
| | % | Total | BG | | 400 | 400 | polypropylene | Candelilla | | Sodium | Immersed |
| | BG | mass | 90 μm-710 μm | BG 32 μm-125 μm | MONO | distearate | glycol | Wax | Sorbitol | Hyaluronate | Compression |
| % | 73 | | 59.00 | 13.65 | 14.5 | 15.2 | 6.0 | 6.0 | .0 | 0.3 | |
| Kg-01- | 73 | 49.425 | 29.5 | 6.825 | 7.25 | 0 | 2.85 | 2.85 | 0 | 0.15 | 4 |
| 22-A | | | | | | | | | | | |
| Kg-01- | 73 | 49.425 | 29.5 | 6.825 | 0 | 7.75 | 3.35 | 1.85 | 0 | 0.15 | 6 |
| 22-B | | | | | | | | | | | |
| Kg-01- | 73 | 49.425 | 29.5 | 6.825 | 7.25 | 0 | 2.85 | 0 | 2.85 | 0.15 | 0 |
| 22-C | | | | | | | | | | | |
| Kg-01- | 73 | 49.425 | 29.5 | 6.825 | 0 | 7.75 | 3.35 | 0 | 1.85 | 0.15 | 0 |
| 22-D | | | | | | | | | | | |

TABLE 6

| | | | | | | PEG | | | | poly- | | |
|---|---|---|---|---|---|---|---|---|---|---|---|---|
| | | | | | PEG | 400 | poly- | | poly- | capro- | | |
| | % | Total | BG | BG | 400 | distea | capro- | Candelilla | propylene | lactone | Sodium | Immersed |
| | BG | mass | 90 μm-710 μm | 32 μm-125 μm | MONO | tearate | lactone | Wax | glycol | diol | Hyaluronate | Compression |
| Kg-01- | 73 | 98.85 | 59 | 13.65 | 14.5 | 0 | 0 | 5.7 | 5.7 | 0 | 0.3 | 6 |
| 23-A | | | | | | | | | | | | |
| Kg-01- | 73 | 49.425 | 29.5 | 6.825 | 7.25 | 0 | 0 | 0 | 2.85 | 2.85 | 0.15 | 4 |
| 23-C-1 | | | | | | | | | | | | |
| Kg-01- | 73 | 49.425 | 29.5 | 6.825 | 0 | 7.75 | 0 | 0 | 3.35 | 1.85 | 0.15 | 4 |
| 23-C-2 | | | | | | | | | | | | |
| Kg-01- | 73 | 49.425 | 29.5 | 6.825 | 7.25 | 0 | 0 | 0 | 2.85 | 2.85 | 0.15 | 5 |
| 23-C-3 | | | | | | | | | | | | |
| Kg-01- | 73 | 49.425 | 29.5 | 6.825 | 7.25 | 0 | 2.85 | 0 | 2.85 | 0 | 0.15 | 0 |
| 23-D-1 | | | | | | | | | | | | |
| Kg-01- | 73 | 49.425 | 29.5 | 6.825 | 0 | 7.75 | 1.85 | 0 | 3.35 | 0 | 0.15 | 0 |
| 23-D-2 | | | | | | | | | | | | |

TABLE 7

| | | | | | PEG | | Glyceryl | | Poly- | | |
|---|---|---|---|---|---|---|---|---|---|---|---|
| | | Total | | BG | 400 | Candelilla | Mono- | Sesame | caprolactone | Sodium | Immersed |
| | % BG | mass | BG 90 μm-710 μm | 32 μm-125 μm | MONO | Wax | stearate | Oil | diol | Hyaluronate | Compression |
| Kg-01- | 73 | 49.9 | 29.5 | 7 | 7.25 | 0 | 3 | 3 | 0 | 0.15 | 4 |
| 25-S-2 | | | | | | | | | | | |
| Kg-01- | 73 | 49.9 | 29.5 | 7 | 7.25 | 3 | 0 | 3 | 0 | 0.15 | 4 |
| 25-S-4 | | | | | | | | | | | |
| Kg-01- | 73 | 49.9 | 29.5 | 7 | 7.25 | 0 | 0 | 3 | 3 | 0.15 | 2 |
| 25-S-5 | | | | | | | | | | | |

TABLE 8

| | | | | | | | | | poly- | | |
|---|---|---|---|---|---|---|---|---|---|---|---|
| | % | Total | BG | BG | PEG 400 | Candelilla | Glyceryl | Coconut | caprolactone | Sodium | Immersed |
| | BG | mass | 90 μm-710 μm | 32 μm-125 μm | MONO | Wax | Monostearate | Oil | diol | Hyaluronate | Compression |
| RK-02- | 73 | 49.9 | 29.5 | 7 | 7.25 | 0 | 3 | 3 | 0 | 0.15 | 2 |
| 3-D2 | | | | | | | | | | | |
| RK-02- | 73 | 49.9 | 29.5 | 7 | 7.25 | 3 | 0 | 3 | 0 | 0.15 | 2 |
| 3-D4 | | | | | | | | | | | |
| RK-02- | 73 | 49.9 | 29.5 | 7 | 7.25 | 0 | 0 | 3 | 3 | 0.15 | 1 |
| 3-D5 | | | | | | | | | | | |

TABLE 9

| | | | | | PEG | | | | poly- | | |
|---|---|---|---|---|---|---|---|---|---|---|---|
| | % | Total | BG | BG | 400 | Candelilla | Glyceryl | Propylene | caprolactone | Sodium | Immersed |
| | BG | mass | 90 μm-710 μm | 32 μm-125 μm | MONO | Wax | Monostearate | Glycol | diol | Hyaluronate | Compression |
| RK-02- | 73 | 49.9 | 29.5 | 7 | 7.25 | 0 | 3 | 3 | 0 | 0.15 | 2 |
| 3-E2 | | | | | | | | | | | |
| RK-02- | 73 | 49.9 | 29.5 | 7 | 7.25 | 3 | 0 | 3 | 0 | 0.15 | 3 |
| 3-E4 | | | | | | | | | | | |
| RK-02- | 73 | 49.9 | 29.5 | 7 | 7.25 | 0 | 0 | 3 | 3 | 0.15 | 2 |
| 3-E5 | | | | | | | | | | | |

TABLE 10

| | | | | | | Poly- | | | | | | |
|---|---|---|---|---|---|---|---|---|---|---|---|---|
| | | | | | | capro- | poly- | poly- | | | | |
| | | Total | Bio- | Bio- | PEG | lactone | caprolactone | caprolactone | Glyceryl | Ses- | | Immersed |
| | % | sample | glass | glass | 400 | 10k | diol | diol | Mono- | ame | Sodium | Com- |
| | BG | mass | 90 μm-710 μm | 32 μm-125 μm | MONO | mw | Diol | Triol | stearate | Oil | Hyaluronate | pression |
| Kg-01- | 73 | 49.9 | 29.5 | 7 | 7.25 | 0 | 3 | 3 | 0 | 0 | 0.15 | 2 |
| 27-A1 | | | | | | | | | | | | |
| Kg-01- | 73 | 49.9 | 29.5 | 7 | 0 | 0 | 3 | 10.25 | 0 | 0 | 0.15 | 2 |
| 27-A2 | | | | | | | | | | | | |
| Kg-01- | 73 | 49.9 | 29.5 | 7 | 0 | 0 | 5 | 8.25 | 0 | 0 | 0.15 | 2 |
| 27-A3 | | | | | | | | | | | | |
| Kg-01- | 73 | 49.9 | 29.5 | 7 | 7.25 | 3 | 0 | 3 | 0 | 0 | 0.15 | 0 |
| 27-B1 | | | | | | | | | | | | |
| Kg-01- | 73 | 49.9 | 29.5 | 7 | 0 | 3 | 0 | 10.25 | 0 | 0 | 0.15 | 0 |
| 27-B2 | | | | | | | | | | | | |
| Kg-01- | 73 | 49.9 | 29.5 | 7 | 0 | 5 | 0 | 8.25 | 0 | 0 | 0.15 | 0 |
| 27-B3 | | | | | | | | | | | | |

TABLE 11

| | | | | | PEG | Poly | | | |
|---|---|---|---|---|---|---|---|---|---|
| | % | Total | BG | | 400 | propylene | Glyceryl | Sodium | Immersed |
| | BG | mass | 90 μm-710 μm | BG 32 μm-125 μm | MONO | glycol | Monostearate | Hyaluronate | Compression |
| Kg-01- | 73 | 47.6 | 28 | 6.5 | 7.25 | 2.85 | 2.85 | 0.15 | 5 |
| 28-A | | | | | | | | | |

TABLE 12

| | | | | | | | | poly- | | | |
|---|---|---|---|---|---|---|---|---|---|---|---|
| | | | | | PEG | | Glyceryl | capro- | | | |
| | % | Total | BG | BG | 400 | Candelilla | Mono- | lactone | | Sodium | Immersed |
| | BG | mass | 90 μm-710 μm | 32 μm-125 μm | MONO | Wax | stearate | triol | diol | Hyaluronate | Compression |
| RK-02- | 73 | 49.9 | 29.5 | 7 | 7.25 | 0 | 3 | 3 | 0 | 0.15 | 4 |
| 4-F2 | | | | | | | | | | | |
| RK-02- | 73 | 49.9 | 29.5 | 7 | 7.25 | 3 | 0 | 3 | 0 | 0.15 | 3 |
| 4-F4 | | | | | | | | | | | |
| RK-02- | 73 | 49.9 | 29.5 | 7 | 7.25 | 0 | 0 | 3 | 3 | 0.15 | 3 |
| 4-F5 | | | | | | | | | | | |

TABLE 13

| | | Total | BG | BG | Bioglass | PEG 400 | Poly propylene | Glyceryl | Sodium | Immersed |
|---|---|---|---|---|---|---|---|---|---|---|
| | % BG | mass | 90 μm-710 μm | 32 μm-125 μm | 2-5 mm | Monostearate | glycol | Monostearate | Hyaluronate | Compression |
| Kg-01- | 66 | 47.6 | | 7.75 | 23.81 | 7.25 | 2.85 | 2.85 | 0.15 | 0 |
| 29-A | | | | | | | | | | |

TABLE 14

| | | | | | | | | Poly | | | | |
|---|---|---|---|---|---|---|---|---|---|---|---|---|
| | | | | | | | | ethylene | | | | |
| | | | | | | Mineral Oil | Mineral | glycol | | | Sodium | Immersed |
| | % | Total | BG | BG | PEG 400 | High | Oil Low | DiMethyl | PEG- | Glyceryl | Hyalu- | Com- |
| | BG | mass | 90 μm-710 μm | 32 μm-125 μm | MONO | Viscosity | Viscosity | Ether | 8 | Monostearate | ronate | pression |
| Kg-01- | 73 | 49.9 | 29.5 | 7 | 7.25 | 3 | 0 | 0 | 0 | 3 | 0.15 | 4 |
| 30-E | | | | | | | | | | | | |
| Kg-01- | 73 | 49.9 | 29.5 | 7 | 7.25 | 0 | 3 | 0 | 0 | 3 | 0.15 | 3 |
| 30-F | | | | | | | | | | | | |
| Kg-01- | 73 | 49.9 | 29.5 | 7 | 7.25 | 0 | 0 | 3 | 0 | 3 | 0.15 | 2 |
| 30-G | | | | | | | | | | | | |
| Kg-01- | 73 | 49.9 | 29.5 | 7 | 7.25 | 0 | 0 | 0 | 3 | 3 | 0.15 | 2 |
| 30-H | | | | | | | | | | | | |

TABLE 15

| | | | | | | | | | Poly | | | |
|---|---|---|---|---|---|---|---|---|---|---|---|---|
| | | | | | | | Mineral | | ethylene | | | |
| | | | | | PEG | | Oil | Mineral | glycol | | | |
| | % | Total | BG | BG | 400 | Candelilla | High | Oil Low | DiMethyl | | Sodium | Immersed |
| | BG | mass | 90 μm-710 μm | 32 μm-125 μm | MONO | Wax | Viscosity | Viscosity | Ether | PEG-8 | Hyaluronate | Compression |
| Kg-01- | 73 | 49.9 | 29.5 | 7 | 7.25 | 3 | 3 | 0 | 0 | 0 | 0.15 | 3 |
| 30-M | | | | | | | | | | | | |
| Kg-01- | 73 | 49.9 | 29.5 | 7 | 7.25 | 3 | 0 | 3 | 0 | 0 | 0.15 | 2 |
| 30-N | | | | | | | | | | | | |
| Kg-01- | 73 | 49.9 | 29.5 | 7 | 7.25 | 3 | 0 | 0 | 3 | 0 | 0.15 | 1 |
| 30-O | | | | | | | | | | | | |
| Kg-01- | 73 | 49.9 | 29.5 | 7 | 7.25 | 3 | 0 | 0 | 0 | 3 | 0.15 | 3 |
| 30-P | | | | | | | | | | | | |

TABLE 16

| | | | | | | | | Poly ethylene | | | | |
|---|---|---|---|---|---|---|---|---|---|---|---|---|
| | | | | | PEG | Mineral Oil | Mineral | glycol | | Poly- | Sodium | Immersed |
| | % | Total | BG | BG | 400 | High | Oil Low | DiMethyl | PEG- | caprolactone | Hyalu- | Com- |
| | BG | mass | 90 μm-710 μm | 32 μm-125 μm | MONO | Viscosity | Viscosity | Ether | 8 | diol | ronate | pression |
| Kg-01- | 73 | 49.9 | 29.5 | 7 | 7.25 | 3 | 0 | 0 | 0 | 3 | 0.15 | 1 |
| 30-Q | | | | | | | | | | | | |
| Kg-01- | 73 | 49.9 | 29.5 | 7 | 7.25 | 0 | 3 | 0 | 0 | 3 | 0.15 | 1 |
| 30-R | | | | | | | | | | | | |
| Kg-01- | 73 | 49.9 | 29.5 | 7 | 7.25 | 0 | 0 | 3 | 0 | 3 | 0.15 | 1 |
| 30-S | | | | | | | | | | | | |
| Kg-01- | 73 | 49.9 | 29.5 | 7 | 7.25 | 0 | 0 | 0 | 3 | 3 | 0.15 | 3 |
| 30-T | | | | | | | | | | | | |

TABLE 17

| | | | | | PEG | | | Poly- | | |
|---|---|---|---|---|---|---|---|---|---|---|
| | % | Total | BG | BG | 400 | Candelilla | Propylene | caprolactone | Sodium | Immersed |
| | BG | mass | 90 μm-710 μm | 32 μm-125 μm | MONO | Wax | Glycol | diol | Hyaluronate | Compression |
| RK-02- | 73 | 99.8 | 59 | 14 | 14.5 | 6 | 6 | 0 | 0.3 | 0 |
| 7-E4 | | | | | | | | | | |
| RK-02- | 73 | 99.8 | 59 | 14 | 14.5 | 0 | 6 | 6 | 0.3 | 0 |
| 7-E5 | | | | | | | | | | |

TABLE 18

| | | | | | PEG | | | PEG | poly- | | |
|---|---|---|---|---|---|---|---|---|---|---|---|
| | | Total | BG | BG | 400 | Candelilla | Glyceryl | 200 | caprolactone | Sodium | Immersed |
| | % BG | mass | 90 μm-710 μm | 32 μm-125 μm | MONO | Wax | Monostearate | adipate | diol | Hyaluronate | Compression |
| Kg-01- | 73 | 49.9 | 29.5 | 7 | 7.25 | 0 | 3 | 3 | 0 | 0.15 | 3 |
| 31-W | | | | | | | | | | | |
| Kg-01- | 73 | 49.9 | 29.5 | 7 | 7.25 | 3 | 0 | 3 | 0 | 0.15 | 2 |
| 31-Y | | | | | | | | | | | |
| Kg-01- | 73 | 49.9 | 29.5 | 7 | 7.25 | 0 | 0 | 3 | 3 | 0.15 | 3 |
| 31-Z | | | | | | | | | | | |

TABLE 19

| | % | Total | | BG | PEG 400 | | Propylene | PCL | Sodium | Immersed |
|---|---|---|---|---|---|---|---|---|---|---|
| | BG | mass | BG 90 μm-710 μm | 32 μm-125 μm | MONO | PEG 400 DI | Glycol | diol | Hyaluronate | Compression |
| Kg-01- | 73 | 49.9 | 29.5 | 7 | | 7.25 | 3 | 3 | 0.15 | 0 |
| 34-E51 | | | | | | | | | | |
| Kg-01- | 73 | 49.9 | 29.5 | 7 | 2 | 5.25 | 3 | 3 | 0.15 | 2 |
| 34-E52 | | | | | | | | | | |
| Kg-01- | 73 | 49.85 | 29.5 | 7 | 3.6 | 3.6 | 3 | 3 | 0.15 | 2 |
| 34-E53 | | | | | | | | | | |

TABLE 20

| | % | Total | BG | BG | PCL | propylene | Sodium | Immersed |
|---|---|---|---|---|---|---|---|---|
| | BG | mass | 90 μm-710 μm | 32 μm-125 μm | diol | glycol | Hyaluronate | Compression |
| Kg-01-35-10 | 74 | 49.95 | 29.5 | 7 | 10.3 | 3 | 0.15 | 1 |

TABLE 21

| | | | | | | | Name of | |
|---|---|---|---|---|---|---|---|---|
| | % | Total | BG | BG | Propyleme | Variable | Variable | Immersed |
| | BG | mass | 90 μm-710 μm | 32 μm-125 μm | Glycol | Component | Component | Compression |
| ZT-02-19-4 | 73 | 49.7 | 29.5 | 7 | 5.1 | 8.1 | Ethylene | 0 |
| | | | | | | | Glycol | |
| | | | | | | | Monostearate | |
| ZT-02-19-5 | 73 | 49.7 | 29.5 | 7 | 5.1 | 8.1 | Propylene | 0 |
| | | | | | | | Glycol | |
| | | | | | | | Stearate | |
| ZT-02-19-6 | 73 | 49.7 | 29.5 | 7 | 5.1 | 8.1 | Brij 2 | 2 |
| ZT-02-19-7 | 73 | 49.7 | 29.5 | 7 | 5.1 | 8.1 | Brij 10 | 0 |
| ZT-02-19-8 | 73 | 49.7 | 29.5 | 7 | 5.1 | 8.1 | Brij 20 | 0 |
| ZT-02-19-9 | 73 | 49.7 | 29.5 | 7 | 5.1 | 8.1 | Brij 100 | 1 |
| ZT-02-19-1 | 73 | 49.7 | 29.5 | 7 | 5.1 | 8.1 | PEG 6000 | 0 |
| | | | | | | | Distearate | |
| ZT-02-19-2 | 73 | 49.7 | 29.5 | 7 | 5.1 | 8.1 | Sorbitan | 0 |
| | | | | | | | Monostearate | |
| ZT-02-19-3 | 73 | 49.7 | 29.5 | 7 | 5.1 | 8.1 | Cetyl Alcohol | 0 |

TABLE 22

| | | | | | | | Name of | |
|---|---|---|---|---|---|---|---|---|
| | % | Total | BG | BG | PEG | Variable | Variable | Immersed |
| | BG | mass | 90 μm-710 μm | 32 μm-125 μm | Adipate | Component | Component | Compression |
| ZT-02-20-A1 | 73 | 49.7 | 29.5 | 7 | 5.1 | 8.1 | PEG 6000 | 3 |
| | | | | | | | Distearate | |
| ZT-02-20-A6 | 73 | 49.7 | 29.5 | 7 | 5.1 | 8.1 | Brij 2 | 2 |
| ZT-02-20-A7 | 73 | 49.7 | 29.5 | 7 | 5.1 | 8.1 | Brij 10 | 3 |
| ZT-02-20-A8 | 73 | 49.7 | 29.5 | 7 | 5.1 | 8.1 | Brij 20 | 3 |
| ZT-02-20-A9 | 73 | 49.7 | 29.5 | 7 | 5.1 | 8.1 | Brij 100 | 2 |
| ZT-02-20-A2 | 73 | 49.7 | 29.5 | 7 | 5.1 | 8.1 | Sorbitan | 0 |
| | | | | | | | Monostearate | |
| ZT-02-20-A3 | 73 | 49.7 | 29.5 | 7 | 5.1 | 8.1 | Cetyl Alcohol | 0 |
| ZT-02-20-A4 | 73 | 49.7 | 29.5 | 7 | 5.1 | 8.1 | Ethylene Glycol | 0 |
| | | | | | | | Monostearate | |
| ZT-02-20-A5 | 73 | 49.7 | 29.5 | 7 | 5.1 | 8.1 | Propylene | 0 |
| | | | | | | | Glycol Stearate | |
| ZT-02-20-A10 | 73 | 49.7 | 29.5 | 7 | 5.1 | 8.1 | Calcium Stearate | 0 |

TABLE 23

| | | | | | | | Name of | |
|---|---|---|---|---|---|---|---|---|
| | % | Total | BG | BG | Butylene | Variable | Variable | Immersed |
| | BG | mass | 90 μm-710 μm | 32 μm-125 μm | Glycol | Component | Component | Compression |
| ZT-02-20-B4 | 73 | 50.9 | 29.5 | 7 | 6.3 | 8.1 | Ethylene | 3 |
| | | | | | | | Glycol | |
| | | | | | | | Monostearate | |
| ZT-02-20-B5 | 73 | 51.7 | 29.5 | 7 | 7.1 | 8.1 | Propylene | 2 |
| | | | | | | | Glycol Stearate | |
| ZT-02-20- | 73 | 48.8 | 29.5 | 7 | 8.3 | 4 | Propylene | 2 |
| MB5 | | | | | | | Glycol Stearate | |
| ZT-02-20-B6 | 73 | 49.7 | 29.5 | 7 | 5.1 | 8.1 | Brij 2 | 4 |
| ZT-02-20-B7 | 73 | 49.7 | 29.5 | 7 | 5.1 | 8.1 | Brij 10 | 2 |
| ZT-02-20-B8 | 73 | 49.7 | 29.5 | 7 | 5.1 | 8.1 | Brij 20 | 9 |
| ZT-02-20-B9 | 73 | 50.2 | 29.5 | 7 | 5.6 | 8.1 | Brij 100 | 2 |
| ZT-02-20-B1 | 73 | 49.7 | 29.5 | 7 | 5.1 | 8.1 | PEG 6000 | 0 |
| | | | | | | | Distearate | |
| ZT-02-20-B2 | 73 | 49.7 | 29.5 | 7 | 5.1 | 8.1 | Sorbitan | 0 |
| | | | | | | | Monostearate | |
| ZT-02-20-B3 | 73 | 49.7 | 29.5 | 7 | 5.1 | 8.1 | Cetyl Alcohol | 0 |
| ZT-02-20- | 73 | 49.7 | 29.5 | 7 | 5.1 | 8.1 | Calcium | 0 |
| B10 | | | | | | | Stearate | |

TABLE 24

| | | | | | | | Name of | |
|---|---|---|---|---|---|---|---|---|
| | % | Total | BG | BG | Hexylene | Variable | Variable | Immersed |
| | BG | mass | 90 μm-710 μm | 32 μm-125 μm | Glycol | Component | Component | Compression |
| ZT-02-20- | 73 | 50.2 | 29.5 | 7 | 5.6 | 8.1 | Sorbitan | 0 |
| H2 | | | | | | | Monostearate | |
| ZT-02-20- | 73 | 50.7 | 29.5 | 7 | 6.1 | 8.1 | Ethylene | 1 |
| H4 | | | | | | | Glycol | |
| | | | | | | | Monostearate | |
| ZT-02-20- | 73 | 49.7 | 29.5 | 7 | 5.1 | 8.1 | Propylene | 1 |
| H5 | | | | | | | Glycol Stearate | |
| ZT-02-20- | 73 | 49.7 | 29.5 | 7 | 5.1 | 8.1 | Brij 2 | 1 |
| H6 | | | | | | | | |
| ZT-02-20- | 73 | 49.7 | 29.5 | 7 | 5.1 | 8.1 | Brij 10 | 1 |
| H7 | | | | | | | | |
| ZT-02-20- | 73 | 49.7 | 29.5 | 7 | 5.1 | 8.1 | Brij 100 | 1 |
| H9 | | | | | | | | |
| ZT-02-20- | 73 | 49.7 | 29.5 | 7 | 5.1 | 8.1 | PEG 6000 | 0 |
| H1 | | | | | | | Distearate | |
| ZT-02-20- | 73 | 49.7 | 29.5 | 7 | 5.1 | 8.1 | Cetyl Alcohol | 0 |
| H3 | | | | | | | | |
| ZT-02-20- | 73 | 49.7 | 29.5 | 7 | 5.1 | 8.1 | Brij 20 | 1 |
| H8 | | | | | | | | |
| ZT-02-20- | 73 | 49.7 | 29.5 | 7 | 5.1 | 8.1 | Calcium | 0 |
| H10 | | | | | | | Stearate | |

TABLE 25

| | | | | | | | PEG | | Propylene | Ethylene | |
|---|---|---|---|---|---|---|---|---|---|---|---|
| | % | Total | BG | BG | Hexylene | Cetyl | 6000 | PEG | Glycol | Glycol | Immersed |
| | BG | mass | 90 μm-710 μm | 32 μm-125 μm | Glycol | Alcohol | Distearate | Adipate | Stearate | Monostearate | Compression |
| ZT-02- | 73 | 49.7 | 29.5 | 7 | 7.2 | 0 | 6 | 0 | 0 | 0 | 1 |
| 23-M1H1 | | | | | | | | | | | |
| ZT-02- | 73 | 49.7 | 29.5 | 7 | 9 | 0 | 4.2 | 0 | 0 | 0 | 0 |
| 23-M2H1 | | | | | | | | | | | |
| ZT-02- | 73 | 49.7 | 29.5 | 7 | 7.2 | 6 | 0 | 0 | 0 | 0 | 0 |
| 23-M1H3 | | | | | | | | | | | |
| ZT-02- | 73 | 49.7 | 29.5 | 7 | 9 | 4.2 | 0 | 0 | 0 | 0 | 0 |
| 23-M2H3 | | | | | | | | | | | |
| ZT-02- | 73 | 49.7 | 29.5 | 7 | | 0 | 0 | 7.2 | 0 | 6 | 0 |
| 23-M1A4 | | | | | | | | | | | |
| ZT-02- | 73 | 49.7 | 29.5 | 7 | | 0 | 0 | 9 | 0 | 4.2 | 0 |
| 23-M2A4 | | | | | | | | | | | |
| ZT-02- | 73 | 49.7 | 29.5 | 7 | | 0 | 0 | 7.2 | 6 | 0 | 0 |
| 23-M1A5 | | | | | | | | | | | |

TABLE 26

| | % | Total | BG | BG | | | Propylene | Immersed |
|---|---|---|---|---|---|---|---|---|
| | BG | mass | 90 μm-710 μm | 32 μm-125 μm | Brij 20 | Brij 100 | Glycol | Compression |
| ZT-02-24-1 | 73 | 49.7 | 29.5 | 7 | 0 | 6 | 7.2 | 2 |
| ZT-02-24-2 | 73 | 49.7 | 29.5 | 7 | 8.1 | 0 | 5.1 | 9 |

TABLE 27

| | % | Total | BG | BG | | | Propylene | Immersed |
|---|---|---|---|---|---|---|---|---|
| | BG | mass | 90 μm-710 μm | 32 μm-125 μm | Brij 20 | Brij 100 | Glycol | Compression |
| Kg-01-37-1 | 73 | 49.7 | 29.5 | 7 | 4 | 3 | 6.2 | 1 |

TABLE 28

| | % | Total | BG | BG | | Propylene | Sodium | Immersed |
|---|---|---|---|---|---|---|---|---|
| | BG | mass | 90 μm-710 μm | 32 μm-125 μm | Brij 20 | Glycol | Hyaluronate | Compression |
| Kg-01-37-6 | 73 | 49.85 | 29.5 | 7 | 8.1 | 5.1 | 0.15 | 8 |
| Kg-01-37-7 | 73 | 49.75 | 29.5 | 7 | 8.1 | 5.1 | 0.05 | 8 |
| Kg-01-37-8 | 72 | 50.85 | 29.5 | 7 | 8.1 | 6.1 | 0.15 | 8 |

Overall the irrigation data shows that samples can be manipulated in an aqueous environment without migrating, washing away or being displaced from the site.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1. An irrigation resistant bone repair composition comprising:
a biocompatible or bioactive bone repair material, and
at least one non-ionic surfactant, wherein the non-ionic surfactant is not a non-random poly(oxyalkylene) block copolymer.

2. The bone repair composition of claim 1, wherein the weight ratio of the at least one non-ionic surfactant is 1%-99% relative to the weight of the bone repair composition.

3. The bone repair composition of claim 1, wherein the weight ratio of the at least one non-ionic surfactant is 1%-20% relative to the weight of the bone repair composition.

4. The bone repair composition of claim 1, wherein the weight ratio of the at least one non-ionic surfactant is 20%-30% relative to the weight of the bone repair composition.

5. The bone repair composition of claim 1, wherein the weight ratio of the at least one non-ionic surfactant is 30%-40% relative to the weight of the bone repair composition.

6. The bone repair composition of claim 1, wherein the weight ratio of the at least one non-ionic surfactant is 40%-50% relative to the weight of the bone repair composition.

7. The bone repair composition of claim 1, wherein the weight ratio of the at least one non-ionic surfactant is 50%-60% relative to the weight of the bone repair composition.

8. The bone repair composition of claim 1, wherein the weight ratio of the at least one non-ionic surfactant is 60%-70% relative to the weight of the bone repair composition.

9. The bone repair composition of claim 1, wherein the weight ratio of the at least one non-ionic surfactant is 70%-80% relative to the weight of the bone repair composition.

10. The bone repair composition of claim 1, wherein the weight ratio of the at least one non-ionic surfactant is 80%-99% relative to the weight of the bone repair composition.

11. The bone repair composition of claim 1, wherein the composition is osteoconductive.

12. The bone repair composition of claim 1, wherein the composition is osteostimulative.

13. The bone repair composition of claim 1, wherein the bone repair material is a bioactive glass or ceramic.

14. The bone repair composition of claim 13, wherein the bioactive glass is melt-derived bioactive glass or sol-gel derived bioactive glass.

15. The bone repair composition of claim 14, wherein the bioactive glass is in the form of a particle, sphere, fiber, mesh, sheet or a combination of these forms.

16. The bone repair composition of claim 14, wherein the bioactive glass comprises about 15-45% CaO, about 30-70% SiO₂, about 0-25% Na₂O, about 0-17% P₂O₅, about 0-10% MgO and about 0-5% CaF₂.

17. The bone repair composition of claim 14, wherein the bioactive glass comprises about 45% SiO₂, about 24.5% CaO, about 6% P₂O₅, and about 2.5% Na₂O.

18. The bone repair composition of claim 15, wherein the size of the bioactive glass particle is in a range from about 0.01 μm to about 5 mm.

19. The bone repair composition of claim 15, wherein the bioactive glass comprises 0-80%<100 μm bioactive glass, 0-80%<500 μm bioactive glass, 0-80% 500-1000 μm bioactive glass, 0-80% 1000-2000 μm bioactive glass, 0-80% 2000-5000 μm bioactive glass, 0-90% 90-710 μm bioactive glass, and 0-90% 32-125 μm bioactive glass.

20. The bone repair composition of claim 15, wherein the bone repair material is one or more particles of bioactive glass coated with a glycosaminoglycan, wherein the glycosaminoglycan is bound to the bioactive glass.

21. The bone repair composition of claim 20, wherein the glycosaminoglycan is selected from the group consisting of heparin, heparan sulfate, chondroitin sulfate, dermatan sulfate, keratan sulfate, and hyaluronic acid.

22. The bone repair composition of claim 1, further comprising at least one element selected from the group consisting of Li, K, Mg, Sr, Ti, Zr, Fe, Co, Cu, Zn, Al, Ag, Ga, P, N, S, F, CI, and I.

23. The bone repair composition of claim 14, wherein the bioactive glass is pretreated in a solution comprising one or more of blood, bone marrow, bone marrow concentrate, bone-morphogenetic proteins, platelet-rich plasma, and osteogenic proteins.

24. The bone repair composition of claim 23, wherein the proteins are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

25. The bone repair composition of claim 1, wherein the composition is in a form of a putty, paste, gel, or waxy solid.

26. The bone repair composition of claim 1, wherein the composition, when implanted into a surgical site, maintains position and does not displace upon irrigation of the surgical site.

27. The bone repair composition of claim 1, wherein the non-ionic surfactant other than the non-random poly(oxyalkylene) block copolymer is selected from the group consisting of fatty alcohols, alkoxylated alcohols, alkoxylated alkylphenols, alkoxylated fatty amides, alkoxylated fatty esters, alkoxylated fatty ethers, alkoxylated sorbitan esters, alkoxylated sorbitan esters, fatty acids, fatty acid esters, polyglycerin fatty acid esters, polyol esters, polyalkylene glycols, alkoxylated organic acids, hydroxyacids or diacids, and copolymers therefrom, and combinations thereof.

28. The bone repair composition of claim 1, wherein the non-ionic surfactant is selected from the group consisting of stearic acid, stearyl alcohol, Ecosurf LF 45, Triton X-100, polyethoxylated tallow amine, poly(ethylene glycol) 400 Monostearate (PEG 400 monostearate), polyethylene glycol lauryl ether (Brij L23), Span 85 (sorbitan trioleate), polysorbate 20, polysorbate 80, glycerol monostearate, PEG coconut triglyceride, PEG 400, PEG 600, sorbitan tristearate, polysorbate 20, polysorbate 80, polyoxyethylene 7 coconut, glyceride, PEG 400 monostearate, PEG 2000 monomethylether, and PEG 400 distearate, polyglyceryl-2 isostearate, polyglyceryl-2 diisostearate, polyglyceryl-4 isostearate, polyglyceryl-6 isostearate, poly(ethylene glycol) 8 stearate (MYRJ S8), polyglyceryl-10 isostearate, polyglyceryl-10 diisostearate, poly(ethylene glycol) 25 propylene glycol stearate (MYRJ S25), poly(ethylene glycol) 400 distearate (PEG 400 distearate), polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, polyglyceryl-10 distearate, polyoxyethylene 7 coconut glyceride (coconut glyceride), polyethylene glycol 2000 monomethyl ether (MME), glyceryl monostearate (monostearin), PEG dimethyl ether (dimethyl polyethylene glycol), PEG 200 adipate (poly(ethylene glycol) 200 adipate, PEG 6000 distearate, sorbitan monostearate, cetyl alcohol, ethylene glycol monostearate, propylene glycol stearate, polyoxyethylene stearyl ether (Brij 2), polyoxyethylene stearyl fatty ether (Brij 10), docosaethylene glycol mono octadecyl ether (Brij 20), polyethylene stearyl ether (Brij 100), polyglycerin fatty acid ester (polyglyceryl-2 isostearate, polyglyceryl-2 diisostearate, polyglyceryl-4 isostearate, polyglyceryl-6 isostearate, polyglyceryl-10 isostearate, polyglyceryl-10 diisostearate, polyglyceryl-4 laurate, polyglyceryl-6 laurate, polyglyceryl-10 laurate, polyglyceryl-10 myristate, polyglyceryl-2 oleate, polyglyceryl-4 oleate, polyglyceryl-6 oleate, polyglyceryl-10 oleate, polyglyceryl-10 stearate, and polyglyceryl-10 distearate.

29. The bone repair composition of claim 1, further comprising an additive selected from the group consisting of a solvent, a linear aliphatic hydrocarbon, straight chain aliphatic hydrocarbon, branched aliphatic hydrocarbon, sugar, poly-saccharide, and hydroxyl terminal polyalkylene oxide, alkoxy terminal polyalkylene oxide, and a low molecular weight biodegradable polymers (MW</=10,000).

30. The bone repair composition of claim 29, wherein the additive is selected from the group consisting of sodium hyaluronate, regenerez, polypropylene glycol 3000 (poly 3000), seasame oil, candelilla wax, carnauba wax, sorbitol (D-Glucitol), polycaprolactone, polycaprolactone diol, coconut oil, propylene glycol, polycaprolactone triol, polycaprolactone 10000 mw, mineral oil high viscosity, mineral oil low viscosity, polyethylene glycol 400, butylene glycol, and hexylene glycol.

31. An irrigation resistant putty or paste including the composition of claim 1 mixed with water, saline, blood, or BMA.

32. The bone repair composition of claim 1, wherein the composition is for treating a bone defect or a bone gap.

33. The bone repair composition claim 1, wherein the composition is for regeneration of hard tissues.

34. A method for treating a bone having a bone gap or a bone defect comprising contacting the bone at or near the site of the bone defect with the bone repair composition of claim 1.

35. A kit comprising:
at least one tube comprising the bone repair composition of claim 1,
a dispensing gun,
an adapter, and
optionally, at least one dispensing tip.

36. The kit of claim 35, wherein the tube comprising the bone repair composition is capped.

37. The kit of claim 35, further comprising a syringe.

38. The kit of claim 35, further comprising at least one of “Y” connector, tube connector, and an aspiration needle.
